(19) United States
(12) Plant Patent Application Publication
(10) Pub. No.: US 2003/0159190 P1
(43) Pub. Date: Aug. 21, 2003

(54) Strawberry plant named 'Camarillo'

(75) Inventors: Amado Q. Amorao, Camarillo, CA (US); Arnoldo Solis JR., Oxnard, CA (US); Michael Ferguson, Chatsworth, CA (US)

Correspondence Address: PENNIE & EDMONDS LLP, 1155 Avenue of the Americas, New York, NY, 10036-2711, US

(73) Assignee: Driscoll Strawberry Associates, Inc.

(21) Appl. No.: 10/077,153

(22) Filed: Feb. 15, 2002

(51) Int. Cl. A01H 5/00

(52) U.S. Cl. PLT/208

(57) ABSTRACT

This invention relates to a new and distinct variety of strawberry named Camarillo. The variety is similar to the varieties Baeza and Ventura. The variety is distinguished from Baeza and Ventura in that Camarillo has a longer fruiting truss, a dark green coloration of the upper side of the leaf, a globose plant habit, even fruit coloration, and absent to small hollow center size.

### 1. BACKGROUND OF THE INVENTION

The new variety originated as a result of a controlled cross between the strawberry plants Baeza (U.S. Plant Pat. No. 11,548) and ‘33X257’ (unpatented variety of Driscoll Strawberry Associates, Inc.) in an ongoing breeding program, and was discovered in Ventura County, California in October, 1997. The original seedling of the new cultivar was asexually propagated by stolons in a nursery in Shasta County, California. Propagules were transplanted to a controlled breeding plot in Ventura County, California, where the variety was identified and selected for further evaluation. Camarillo was subsequently asexually propagated and underwent further testing Ventura County, California for five years. This propagation and testing has demonstrated that the combination of traits disclosed herein which characterize the new variety are fixed and retained true to type through successive generations of asexual reproduction.

### 2. SUMMARY OF THE INVENTION

The present invention relates to a new and distinct variety of strawberry named Camarillo. The variety is botanically identified as Fragaria×ananassa. The new variety is distinguished from other varieties by a number of characteristics as set forth in Tables 1-4.

### 3. COMPARISON TO SIMILAR VARIETIES

The varieties which we believe to be similar to Camarillo from those known to us are Baeza (U.S. Plant Pat. No. 11,548) and Ventura. There are several characteristics of the new variety that are different from, or not possessed by Baeza and Ventura. The new variety has a longer fruiting truss, a dark green coloration of the upper side of the leaf, a globose plant habit, even fruit coloration, and an absent to small hollow center size.

### 4. BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 shows a close-up photo of the whole plant.

FIG. 2 shows the whole plant.

FIG. 3 shows the leaves of the plant.

FIG. 4 shows the upper side and the under side of the flowers.

FIG. 5 shows a close-up of the fruit.

FIG. 6 shows the fruit in longitudinal cross-section.

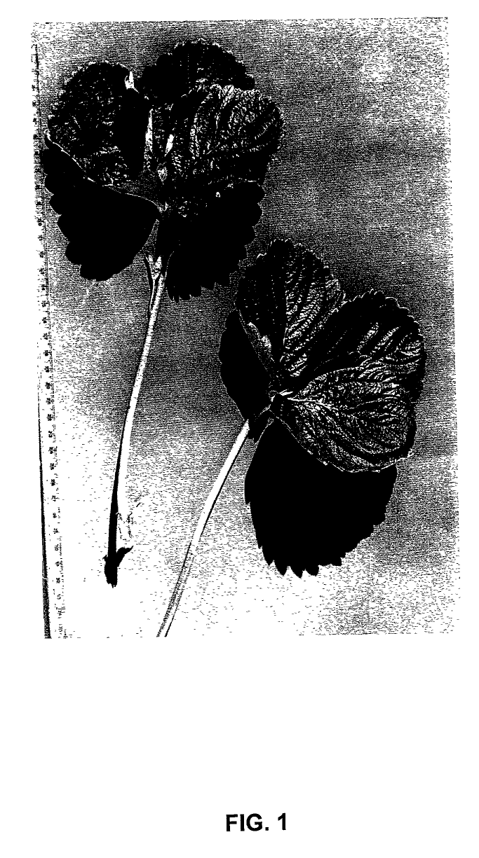

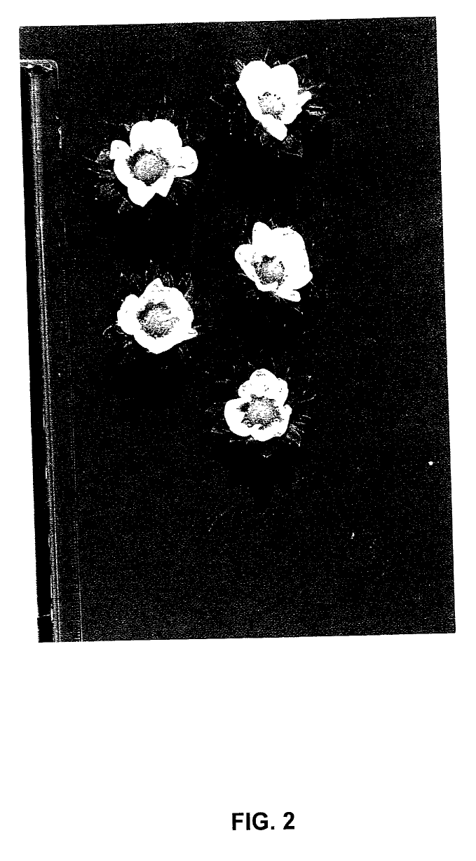

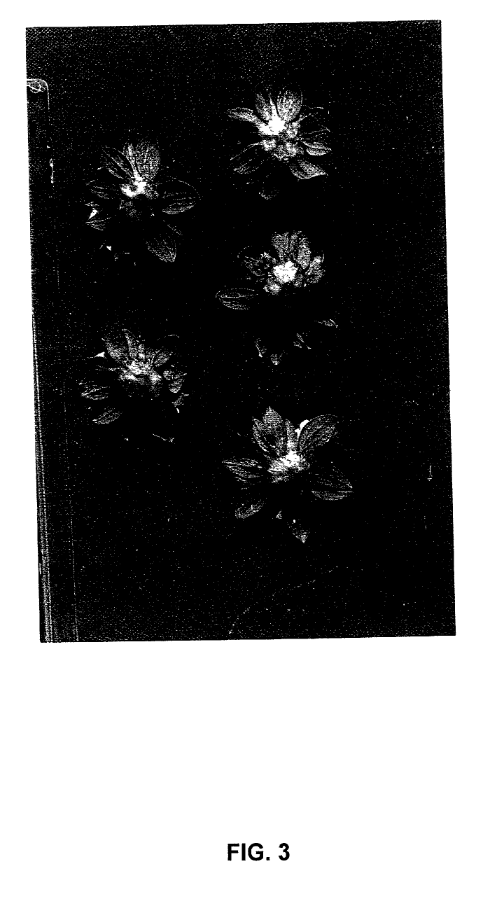

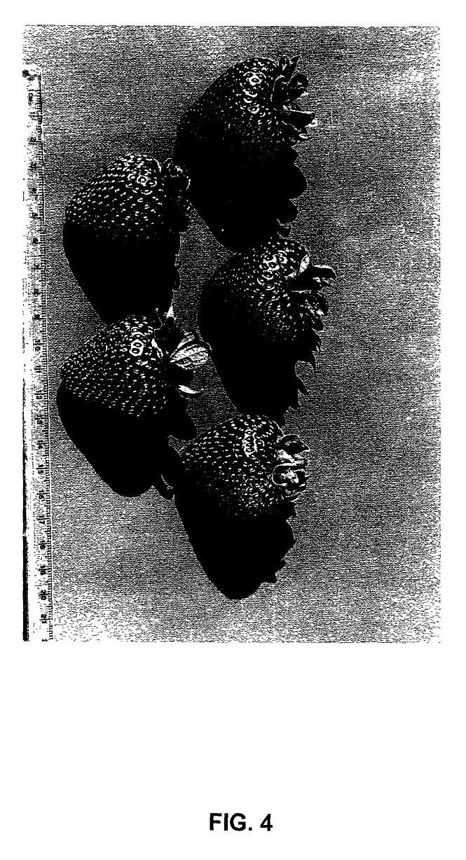

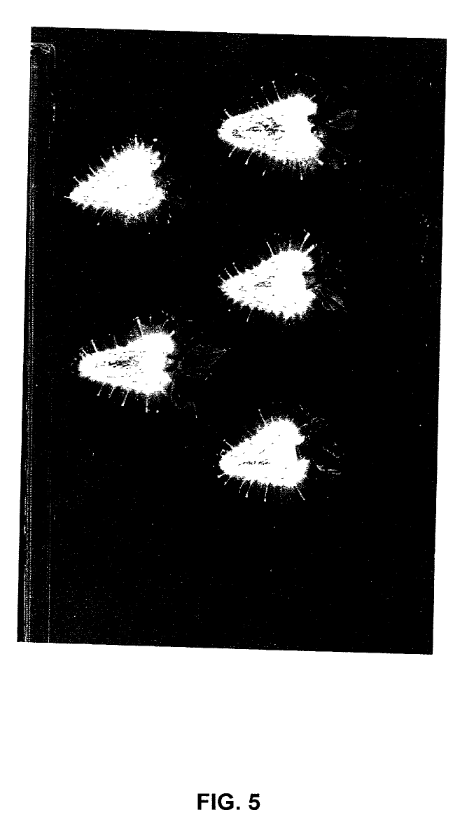

### 5. DESCRIPTION OF THE NEW VARIETY

The following detailed description of the new variety is based upon observations taken of plants and fruit grown in Ventura County, California, U.S.A. Observations of Camarillo, Baeza and Ventura were taken in side by side comparison in 2001. This description is in accordance with UPOV terminology. Color designations, color descriptions, and other phenotypical descriptions may deviate from the stated values and descriptions depending upon variation in environmental, seasonal, climatic and cultural conditions. Colors are described and the most similar color designations are provided from The Royal Horticultural Society (RHS) Colour Chart.

### 5.1 PROPAGATION

The new variety is principally propagated by way of stolons. Although propagation by stolons is presently preferred, other known methods of propagating strawberry plants may be employed.

### 5.2 CHARACTERISTICS OF THE NEW VARIETY

Information on the new variety is presented in Tables 1, 2 and 3. In the tables, the flowers described are secondary flowers except where indicated. The fruit described is the secondary fruit on one year old plants. Fruit and flower measurements are an average of both primary and secondary fruit and flowers.

Table 1 provides a quantitative comparison of the plant and fruit characteristics of the new variety Camarillo compared with characteristics of Baeza and Ventura. Table 2 provides additional information of the plant and fruit characteristics of the new variety Camarillo compared with characteristics of the varieties Baeza and Ventura. Table 3 provides reactions of the new variety to stresses, pests and disease as compared to the varieties Baeza and Ventura. Table 4 provides isozyme characteristics of the new variety as compared to the varieties Baeza and Ventura.

TABLE 1
DETAILED COMPARISON OF
CAMARILLO, BAEZA AND VENTURA

| | CAMARILLO | BAEZA | VENTURA |
|---|---|---|---|
| Plant Characteristics | | | |
| Height of Plant (cm) | 23.3 | 20.8 | 21.0 |
| Spread of Plant (cm) | 42.7 | 38.2 | 38.7 |
| Number of Crowns | 4.8 | 3.0 | 3.3 |
| Leaf Characteristics | | | |
| Terminal Leaflet | 8.2 | 8.5 | 7.5 |
| Length (cm) | | | |
| Terminal Leaflet | 8.2 | 8.7 | 7.5 |
| Width (cm) | | | |
| Terminal Leaflet | 1.0 | 0.98 | .99 |
| Length/Width | | | |
| # Teeth/Terminal | 24.8 | 25.4 | 22.4 |
| Leaflet | | | |
| Color of upper side | dark green | light to | medium green |
| | 147A | medium green | 137A |
| | | 147A | |
| Color of under side | light green | light green | light green |
| | 138B | 138B | 138B |
| Petiole Length (cm) | 15.9 | 14.5 | 14.2 |
| Petiole Color | 149A | 144A | 145A |
| | yellow green | yellow green | yellow green |
| Bract Frequency | 42% | 67% | 50% |
| | mostly double | mostly double | mostly double |
| Stipule Length (cm) | 3.5 | 3.5 | 2.8 |
| Stipule Width (cm) | 1.2 | 1.1 | 1.1 |
| Stolon | | | |
| Diameter at base of | 4.09 | 4.12 | 4.05 |
| last daughter | | | |
| Flower Characteristics | | | |
| Petal Length (cm) | 1.22 | 1.10 | 1.19 |
| Petal Width (cm) | 1.39 | 1.22 | 1.09 |
| Petal Length/Width | 0.88 | 0.90 | 1.09 |
| Ratio | | | |
| Petal color | 155B | | |
| Flower Diameter (cm) | 2.61 | 2.50 | 2.40 |
| Calyx Diameter (cm) | 2.98 | 2.55 | 2.57 |
| Fruiting Truss | | | |
| Length (cm) | 32.0 | 28.5 | 24.8 |
| Fruit Characteristics | | | |
| Fruit Length (cm) | 4.1 | 4.2 | 4.5 |
| Fruit Width (cm) | 4.0 | 3.8 | 4.0 |
| Fruit Length/Width | 1.03 | 1.11 | 1.11 |
| Ratio | | | |
| Average Berry | 21.1 | 21.5 | 24.3 |
| Weight (g) | | | |
| External Color | 46A | 46A | 46A |
| | red | red | red |
| Internal Color | 34B & 155A | 42B & 155D | 44A |
| | orange red & | white & | orange red |
| | white | orange red | |
| Average % brix | 9.26 | 10.38 | 9.27 |
| Brix/Acid Ratio | 12.62 | 12.87 | 12.95 |
| Achene Coloration | 184B and 13B | 13A and 46A | 13B and 45B |
| Marketable Yield in | 410 | 293 | 118 |
| 2001 (g/plant) | | | |

TABLE 2
CHARACTERISTICS OF
CAMARILLO, BAEZA AND VENTURA

| | CAMARILLO | BAEZA | VENTURA |
|---|---|---|---|
| Plant | | | |
| Habit | globose | flat globose | globose to flat |
| | | | globose |
| Density | open | open | medium |
| Vigor | medium | medium | weak to |
| | | | medium |
| Leaf | | | |
| Shape in cross section | concave | concave | slightly |
| | | | concave |
| Interveinal blistering | very strong | strong to very | strong |
| | | strong | |
| Glossiness | medium to | weak | medium |
| | strong | | |
| Number of leaflets | three only | three only | three only |
| Terminal leaflet | revolute to flat | revolute to flat | revolute to flat |
| margin profile | | | |
| Terminal leaflet shape | rounded | obtuse to | rounded |
| of base | | rounded | |
| Terminal leaflet shape | rounded | acute to | obtuse |
| of teeth | | obtuse | |
| Stipule pubescence | sparse | sparse | sparse |
| Petiole pubescence | sparse | very sparse to | sparse |
| | | sparse | |
| Petiole pose of hairs | outwards | outwards | outwards |
| Stolon | | | |
| Amount | few to medium | few to medium | few to medium |
| Anthocyanin | weak to | weak to | medium |
| coloration | medium | medium | |
| Thickness | thick | thick to | medium to |
| | | very thick | thick |
| Pubescence | sparse | medium to | dense |
| Inflorescence | | dense | |
| Position relative | above | level to above | level to above |
| to foliage | | | |
| Diameter of calyx | smaller to same | same size to | smaller |
| relative to | size | larger | |
| corolla on | | | |
| secondary flowers | | | |
| Diameter of inner | same size | same size | same size |
| calyx relative | | | |
| to outer on | | | |
| secondary flowers | | | |
| Spacing of petals | overlapping | overlapping | touching to |
| | | | overlapping |
| Fruiting Truss | | | |
| Attitude at | prostrate | prostrate | semi-erect |
| first picking | | | |
| Fruit | | | |
| Predominant shape | cordate | conical | conical to |
| | | | cordate |
| Difference in shapes | slight | very slight to | slight |
| between primary and | | slight | |
| secondary fruits | | | |
| Band without achenes | absent or very | very narrow | narrow |
| | narrow | to narrow | |
| Unevenness of surface | weak | weak to | weak to |
| | | medium | medium |
| Evenness of color | even | slightly uneven | slightly even |
| uneven | | to even | |
| Glossiness | strong | strong | strong |
| Insertion of achenes | below surface | level to below | below surface |
| | | surface | |
| Insertion of calyx | in a basin | level | in a basin to |
| | | | level |
| Pose of the calyx | spreading | spreading to | reflexed |
| segments | | reflexed | |
| Size of calyx in | smaller | same size to | smaller |
| relation to fruit | | larger | |
| on secondary fruit | | | |
| Adherence of calyx | strong | strong | weak to |
| | | | medium |
| Firmness of flesh | firm | medium to firm | firm |
| Evenness of flesh | slightly uneven | uneven | slightly uneven |
| color | | | |
| Distribution of flesh | marginal and | marginal to | marginal to |
| color | central | central | central |
| Hollow center size | absent to small | large | small |
| Sweetness | medium | medium | medium to |
| | | | strong |
| Texture when tasted | medium | medium | fine |
| Acidity | medium | medium | weak to |
| | | | medium |
| Time of Flowering | mid to late | mid to late | mid to late |
| | August | August | August |
| Harvest Interval in | 9/29-12/22 | 9/29-12/22 | 10/6-12/22 |
| 2001 (Week Ending) | | | |
| Type of Bearing | fully | fully | fully |
| | everbearing | everbearing | everbearing |

TABLE 3
5.3 REACTION TO STRESS, PESTS, AND DISEASE

| | CAMARILLO | BAEZA | VENTURA |
|---|---|---|---|
| Reaction to Stress | | | |
| high pH | moderately | moderately | moderately |
| | resistant | resistant | resistant |
| high soil salt levels | moderately | susceptible | moderately |
| | resistant | | resistant |
| Reaction to Pests | | | |
| Tetranychus urticae | moderately | moderately | moderately |
| | susceptible | susceptible | susceptible |
| Lygus hesperus | susceptible | susceptible | susceptible |
| Reaction To Diseases | | | |
| Botrytis fruit rot | susceptible | susceptible | moderately |
| | | | susceptible |
| Powdery mildew | susceptible | highly | highly |
| | | susceptible | susceptible |
| Verticillium wilt | susceptible | susceptible | susceptible |
| Strawberry Mottle Virus | moderately | moderately | moderately |
| | resistant | resistant | resistant |
| Xanthomonas fragariae | moderately | moderately | moderately |
| | resistant | resistant | resistant |

### 5.5 ISOZYME ANALYSIS

In addition to the morphological description above, the new cultivar Camarillo has been analyzed to obtain an indication of its genetic makeup to provide further means for identifying the new variety and distinguishing it from some other somewhat similar and/or related strawberry varieties. Specifically, leaf samples of Camarillo, Baeza, and Ventura were analyzed by electrophoresis for isozyme patterns of the enzymes phosphoglucoisomerase (“PGI”), leucine aminopeptidase (“LAP”) and phosphoglucomutase (“PGM”). See J. Amer. Soc. Hort. Sci. 106:684-687. Isozyme characterization of the three varieties is presented in Table 4, with the letters representing the banding patterns for each enzyme as designated in the above-identified article.

TABLE 4
ISOZYME ANALYSIS FOR
CAMARILLO, BAEZA AND VENTURA

| Locus | CAMARILLO | BAEZA | VENTURA |
|---|---|---|---|
| PGI | A2 | A1 | A2 |
| LAP | B3 | B3 | B3 |
| PGM | C4 | C3 | C4 |

What is claimed is:

1. A new and distinct variety of strawberry plant, substantially as shown and described.
